(19) United States
(12) Patent Application Publication
(10) Pub. No.: US 2022/0257131 A1
(43) Pub. Date: Aug. 18, 2022

(54) PORTABLE SENSOR SYSTEM WITH MEASURING PATCH

(71) Applicant: OSRAM Opto Semiconductors GmbH, Regensburg (DE)

(72) Inventor: Mathieu RAYER, Augsburg (DE)

(21) Appl. No.: 17/625,792

(22) PCT Filed: Jul. 6, 2020
(86) PCT No.: PCT/EP2020/068970

§ 371 Date: Jan. 10, 2022

(30) Foreign Application Priority Data
Jul. 11, 2019 (DE) 10 2019 118 864.4

(51) Int. Cl. A61B 5/024; A61B 5/145; A61B 5/0537; A61B 5/00

(52) U.S. Cl. CPC A61B 5/02427; A61B 5/02438; A61B 2560/0462; A61B 5/0537; A61B 5/6833; A61B 5/14507

(57) ABSTRACT

A portable system may measure and monitor physiological parameters of a human being by analysis of a body fluid. The system may include a consumable part in the form of a measuring patch to be adhered to the skin. The measuring patch may be equipped with a sensor system configured to provide measured values of physiological parameters by body fluid analysis. The system may further include a body-worn readout unit suitable for continuous use, which is configured to read out and monitor the measured values supplied by the measuring patch. The readout unit is further configured to be worn over the measuring patch adhered to the skin and to read out and monitor the measured values in this position.

### CROSS-REFERENCE TO RELATED APPLICATIONS

The present application is a national stage entry according to 35 U.S.C. § 371 of PCT application No.: PCT/EP2020/068970 filed on Jul. 6, 2020; which claims priority to German Patent Application Serial No.: 10 2019 118 864.4 filed on Jul. 11, 2019; all of which are incorporated herein by reference in their entirety and for all purposes.

### TECHNICAL FIELD

The present disclosure relates to a portable system for measuring and monitoring physiological parameters of a human being by analysis of one of his body fluids, the system comprising:
a consumable in the form of a measuring patch to be adhered to the skin, the measuring patch being equipped with a sensor system configured to provide measured values of physiological parameters by body fluid analysis; and
a body-worn readout unit suitable for continuous use, which is configured up to read out and monitor the measured values provided by the sensor system of the measuring patch.

### BACKGROUND

Such a system is known from the generic article “A soft, wearable microfluidic device for the capture, storage, and colorimetric sensing of sweat” by Koh et al. published in Science Translational Medicine, vol. 8, reference 366ra165, on 23 Nov. 2016.

This well-known system consists of a measuring patch and a smartphone. The measuring patch includes sensors that are able to analyse the sweat of the wearer of the measuring patch for certain ingredients and the pH value. The analysis is based on colourimetry. Photos of the measuring patch in use are taken with the smartphone, which is equipped with a camera. Image processing software installed on the smartphone is able to evaluate the photos taken. In this way, conclusions can be drawn about the concentration of the sweat ingredients and the pH value.

The disadvantage of this well-known system is that the user has to take photos with his smartphone at regular intervals. This represents a considerable effort. In addition, the development of dedicated image processing software capable of correctly interpreting the colours and patterns on the patch is complex. Because of the complexity, such image processing software is also quite error prone.

Fitness wristbands are also known from the state of the art. These can be used to monitor the pulse and the oxygen saturation of the blood during sport. The sensors used there are integrated into the wristband and consist, for example, of one or more light-emitting diodes and an associated photodiode. The light emitted by the light-emitting diodes in the direction of the skin is partly absorbed and partly reflected by the skin. Based on the temporal course of the intensity of the reflected light, conclusions can be drawn about the pulse and the oxygen saturation of the blood.

The disadvantage of these solutions is that they only measure two physiological parameters. This means that it is not possible to make a comprehensive statement about the wearer's state of health or fitness.

An overview of the variety of existing wearable sensors is provided in the article “Wearable sensors: modalities, challenges, and prospects” by J. Heikenfeld et al. published on 21 Jan. 2018 in the journal Lab on a Chip, Vol. 18, No. 2, pages 216-248.

It is an objective to develop the system defined above in such a way that it is simpler and more convenient to use and is suitable for real-time monitoring.

### SUMMARY

The objective may be solved by the fact that the readout unit is further configured to be worn over the measuring patch adhered to the skin and to read out and monitor the measured values in this position.

The fact that the user of the system can place the readout unit over the patch enables a regular and automated exchange of data between the patch and the readout unit. No involvement of the user is required.

In a non-limiting embodiment, the measuring patch may comprise an output for the delivery of the measured values, the readout unit may comprise an input complementary to the output for the reception of the measured values, and the measuring patch and the readout unit may be arranged in such a way that the output and the input are in close proximity to each other when used on humans, and may contact each other in order to form an interface for the transmission of the measured values.

The interface can be an optical and/or electrical interface.

The output and the input can each comprise a complementary contact surface.

In a non-limiting embodiment, an access window is formed in the body of the measuring patch and a sensor device is provided in the readout unit, which is located in use above the access window so that it has direct access to the skin via the access window.

The system may comprise a chemical sensor arrangement for determining the concentration of a substance in the body fluid.

The chemical sensor arrangement can be based on colourimetry, with a colourimetric reagent in the measuring patch and an optical detector in the readout unit.

The optical detector may comprise a light source for illuminating the reagent and a photodiode for detecting the light reflected back from the reagent.

The system may comprise an optoelectronic sensor arrangement, such as for determining pulse rate and/or blood pressure.

The system may also comprise a volumetric sensor arrangement for determining a volume of the body fluid.

In this case, the volumetric sensor arrangement may comprise a microfluidic channel in the measuring patch.

In one embodiment, the system comprises an electrical sensor arrangement for determining the concentration of a substance in the body fluid.

The electrical sensor arrangement may comprise an electrode located in the measuring patch and coated with a reagent.

In one embodiment, the system is equipped with a sensor arrangement for bioelectrical impedance analysis.

The sensor arrangement for bioelectrical impedance analysis may comprise electrodes integrated in the readout unit.

In one embodiment, the system comprises a mechanical pressure sensor for measuring deformations of the skin, which may be integrated in the measuring patch.

The body fluid may be in particular sweat, blood, spit or tears.

### BRIEF DESCRIPTION OF THE DRAWINGS

In the following, some embodiments are described in more detail by way of example with reference to the drawings, wherein:

FIG. 1 shows a wearable system for measuring physiological parameters, which is intended for use on the wrist of a user;

FIG. 2 gives an overview of the variety of physiological parameters that can potentially be measured and monitored with the readout unit and the consumable part of a system;

FIGS. 3A-3B show a first embodiment of a possible sensor system of a measuring system;

FIGS. 4A-4B show a second embodiment of a possible sensor system of a measuring system;

FIGS. 5A-5B show a third embodiment of a possible sensor system of a measuring system; and

FIGS. 6A-6B show a fourth embodiment of a possible sensor system of a measuring system.

### DETAILED DESCRIPTION

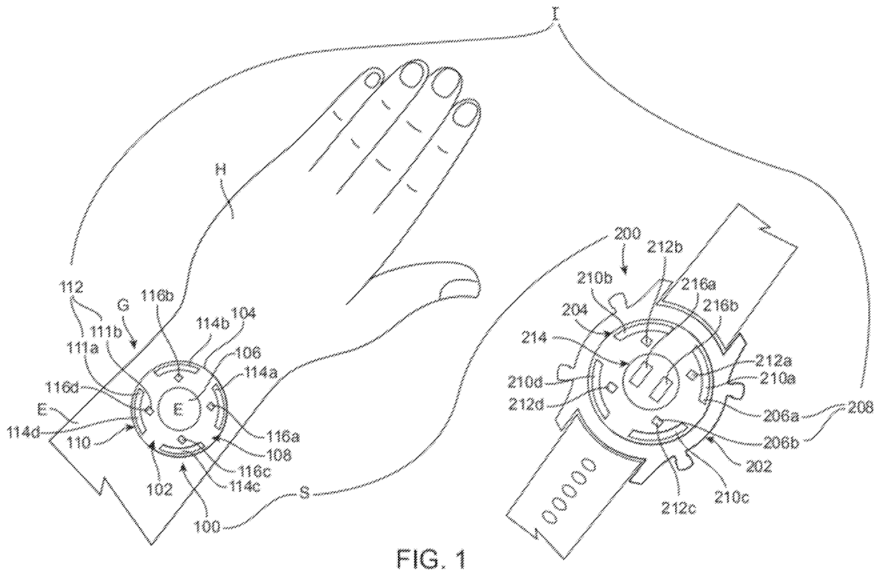

FIG. 1 shows an exemplary embodiment of a portable system S for measuring and monitoring physiological parameters of a human being by analysing one of his body fluids.

The system S stands out, among other things, because it consists of two complementary components 100, 200. The two components 100, 200 work together to measure and monitor physiological parameters of the wearer of the system S. The one component of the system S is thereby a consumable part 100. This consumable part 100, can be attached to the user's body in order to take measurements at the corresponding part of the body. The consumable part is a measuring patch 100 that can be attached to the skin E. The measuring patch 100 has only a short life span of, for example, a few hours, one or more days or a week. As soon as the measuring patch 100 is used up, it is thrown away and replaced by a new measuring patch.

The second component 200 of the wearable system S has a significantly longer life span compared to the sticking patch 100, for example one or two years. This component 200 is an object that is worn on the body by the user. One can therefore also consider the second component 200 as a kind of main or base unit, and the consumable 100 as an accessory of the base unit 200. The base unit 200 may contain the core elements of the measuring system S, such as the evaluation electronics, the user interface and the like.

The consumable part 100 comprises only inexpensive and easily replaceable components of the measuring system S. The base unit 200 is thus designed for frequent and permanent use. In other words, the base unit 200 is a reusable object. In contrast, the consumable part 100 is in particular a disposable object.

The second component 200 is therefore a device that is purchased by a user for long-term use. The consumable 100 is an accessory that is available separately and in larger quantities. It is combined with the device 200 to create its full functionality.

The base unit 200 and the consumable part 100 can also be considered as two related products that complement and interact with each other.

In the measuring system S shown in FIG. 1, the base unit 200 is formed by a wristwatch. FIG. 1 shows the back of the wristwatch 200.

However, the base unit 200 can also be another item designed to be worn on a part of the body. For example, it could be a bracelet, a ring, a necklace, a garment or similar. The exact shape is irrelevant here, as long as the base unit 200 is configured to be worn over the consumable part 100 adhered to the skin E.

In order for the system S to measure and monitor physiological parameters of its wearer, the measuring patch 100 is equipped with a sensor system 102. This sensor system 102 typically comprises several components. The measuring patch 100 is configured such that the sensor system 102 can analyse a body fluid of the wearer. Thus, the measuring patch 100 may include means for receiving or storing one or more body fluids of the wearer.

In the measuring system S shown in FIG. 1, the measuring patch 100 is adhered to the skin E of the wrist G of the hand H of the user. The body fluid analysed by the measuring system S is sweat. More precisely, it is the sweat secreted by the skin E located underneath the measuring patch 100.

Alternatively, the body fluid could also be blood, spit or tears, in which case the measuring patch 100 could be applied to another suitable part of the body. It is also conceivable that the measuring patch 100 analyses not only one, but several body fluids.

The base unit 200 is configured to read out and monitor the measured values provided by the sensor system 102 of the measuring patch 100. The base unit 200 can therefore also be referred to as the readout unit. The readout and monitoring by the readout unit 200 takes place when it is worn over the measuring patch 100 adhered to the skin.

In the measuring system S shown in FIG. 1, the measuring patch 100 is a patch with a circular outer contour 104. The measuring patch 100 is therefore a thin disc. A hole 106 is formed in the centre of the disc. This hole 106 is referred to below as the access window.

The material of the measuring patch 100 can be plastic, paper or a similar material which is well tolerated by the skin and fits well to the skin E. The sensor system 102 is incorporated in this material. The measuring patch 100 has a front side 108 and a back side 110. In use, the front side 108 faces the readout unit 200, whereas the back side 110 contacts the skin E. For better adhesion to the skin, the back side 110 of the measuring patch 100 may be provided with an adhesive.

The front side 108 comprises areas 111a and 111b which together form an output 112 for delivering the measured values to the readout unit 200.

The readout unit 200, i.e. the wristwatch in the present example, comprises a front 202 and a back 204. The front 202 is used for interaction with the user, for example by means of a display.

In use, the back 204 rests on the front side 108 of the measuring patch 100. The back 204 comprises areas 206a and 206b which together form an input 208 for receiving the measured values.

Accordingly, the output 112 of the measuring patch 100 and the input 208 of the wristwatch 200 are in close proximity to each other when used on humans. In the measuring system S shown in FIG. 1, the output 112 and the input 208 make contact with each other and thus form an interface I which is used for transmitting measured values. The input 208 is complementary to the output 112.

The interface I can in particular be an optical and/or electrical interface.

In the measuring system S according to FIG. 1, the sensor system 102 of the measuring patch 100 consists of an electrical sensor arrangement 114a to 114d and a chemical sensor arrangement 116a to 116d.

The electrical sensor arrangement comprises four electrodes 114a, 114b, 114c and 114d. The four electrodes are formed here on the periphery of the measuring patch 100 in the form of circular arcs.

The chemical sensor arrangement comprises four fluid chambers 116a, 116b, 116c and 116D. These fluid chambers are evenly distributed over the measuring patch 100 at the same distance from the centre of the measuring patch 100.

The surfaces or regions 111b of the four fluid chambers 116a to 116d and the surfaces or regions 111a of the electrodes 114a to 114d together form a contact surface 111a, 111b of the output 112 of the measuring patch 100.

The back 204 of the readout unit 200 comprises an electrical readout arrangement 210a to 210d that is complementary to and interacts with the electrical sensor arrangement 114a to 114d of the measuring patch 100. In the present case, the electrical readout arrangement 210a to 210d comprises four readout electrodes arranged at the periphery of the back 204 and having a circular arc shape.

The back 204 also comprises an optical readout arrangement 212a to 212d. This is complementary to and interacts with the chemical sensor arrangement 116a to 116d. The optical readout arrangement 212a to 212d comprises four pairs of diodes 212a, 212b, 212c and 212d not shown in detail in FIG. 1. Each diode pair comprises a light emitting diode (LED) and a light detecting photodiode. The four pairs are equally spaced from the centre of the back 204 and evenly distributed across it. Each pair 212a to 212d is associated with a fluid chamber 116a to 116d.

The surfaces or areas 206b of the four pairs of diodes 212a to 212d and the surfaces or areas 206a of the four electrodes 210a to 210d together form a contact area 206a, 206b of the input 208 which is complementary to the contact area 111a, 111b of the output 112. The two contact surfaces 111a, 111b; 206a, 206b allow the measuring patch 100 and the wristwatch 200 to exchange measurement signals or data.

The access window 106 in the measuring patch 100 allows the wristwatch 200 to directly access the skin E of the wearer of the measurement system S. For this purpose, a sensor device 214 is provided in the centre of the back 204 of the wristwatch 200. The sensor device 214 is located in use above the access window 106. The sensor device 214 has, for example, two sensor elements 216a and 216b, which may be, for example, a light emitting diode and a photodiode.

The measuring patch 100 can be equipped with a wide variety of sensors systems 102 as required. It is also conceivable that there are different types of measuring patches 100 for different applications.

For example, a user can use a different patch when she is exercising than when she is sleeping. For the first case, there is a first type of measuring patch, which is designed as a so-called fitness tracker. For the second case, there is a second type of measuring patch that is equipped with sensors 102 that make it possible to monitor the user's sleep behaviour.

The readout unit 200 is then configured so that its input 208 is compatible with all types of patches.

The readout unit 200 can also be designed in different variants. These can differ in which physiological parameters they monitor.

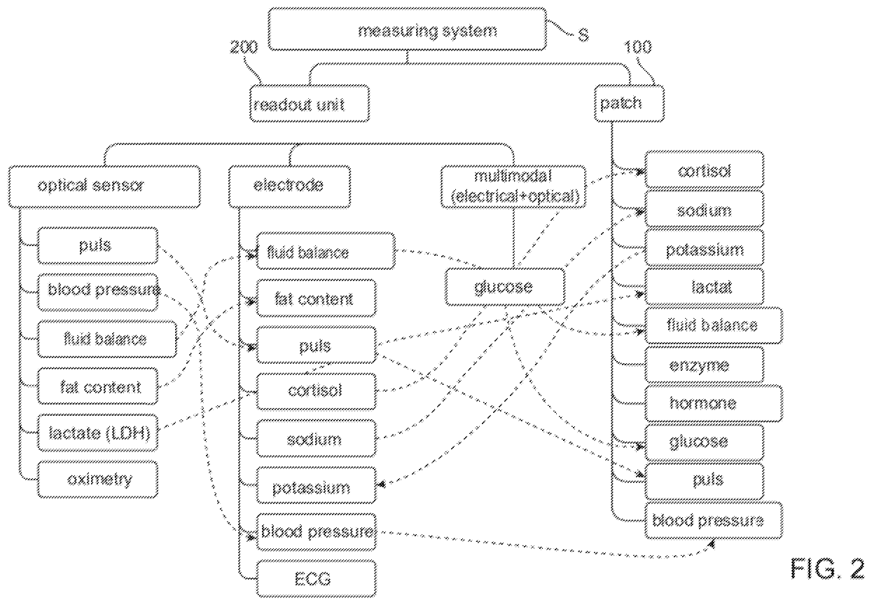

The diagram in FIG. 2 illustrates the multitude of different physiological parameters that can potentially be measured with the portable measuring system S.

For example, optical sensors may be present in the readout unit 200, by means of which the pulse, blood pressure, fluid balance, fat content, lactate content or oxygen saturation of the blood (oximetry) can be measured.

Additionally or alternatively, electrodes may be provided in the readout unit 200 to measure fluid balance, fat content, pulse, cortisol level, sodium level, potassium level or blood pressure, or to record an electrocardiogram (ECG).

If both electrodes and optical sensors are available, it is also conceivable to determine blood sugar (glucose) by means of an electro-optical multimodal measurement.

The sensor system 102 in the measuring patch 100 may be used, for example, to measure cortisol levels, sodium levels, potassium levels, lactate levels, fluid balance, enzyme or hormone identification, sugar (glucose) measurement, pulse measurement, or blood pressure measurement.

The arrows in the diagram in FIG. 2 each connect the same physiological parameters from the readout unit to the measuring patch.

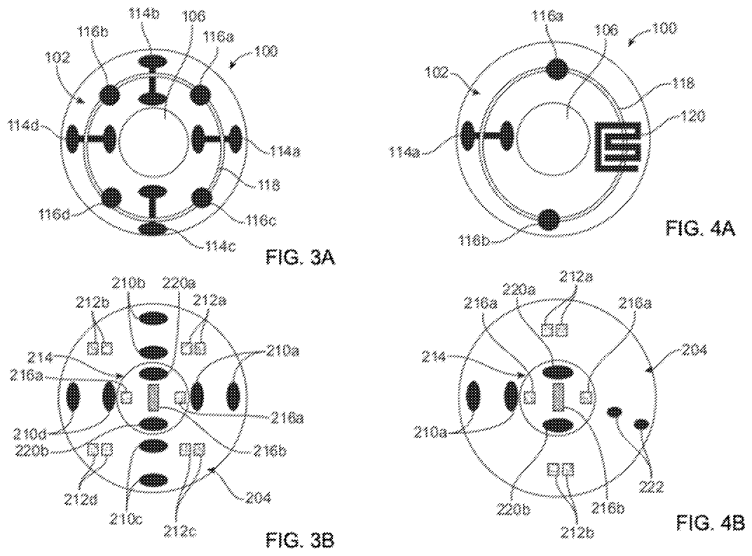

With reference to FIGS. 3A-6B, four possible configurations for the sensor system 102 of the measuring patch 100 and the associated readout arrangements of the readout unit 200 are now described by way of example.

In the variant of FIGS. 3A-3B, the associated measuring system S is capable of determining the concentration of four different substances in the body fluid, determining a volume of excreted body fluid, carrying out a bioelectrical impedance analysis, recording the pulse and blood pressure, and producing an electrocardiogram (ECG).

FIG. 3A schematically shows the design of the measuring patch 100 and FIG. 3B schematically shows the design of the complementary back 204 of the readout unit 200.

There are four chemical sensor arrangements (116a, 212a); (116b, 212b); (116c, 212c) and (116d, 212d), each of which is used to determine the concentration of a different substance in the body fluid. For example, when the body fluid is sweat, the sodium and potassium levels, the pH and also the cortisol level can be determined using the four sensor arrangements. Each chemical sensor arrangement here comprises a fluid chamber 116A to 116D in which a reagent is provided. The reagent provided in each fluid chamber is selected to be responsive to the substance to be detected. Each fluid chamber 116a to 116d is associated with a respective optical detector 212a to 212d. In this case, the chemical sensor arrangements are based on colorimetry. This means that with increasing concentration of the substance to be measured in the respective liquid chamber 116a to 116d, a corresponding increasing discolouration occurs, triggered by the reaction of the reagent with the substance. The discolouration is detected thanks to the optical detectors 212a to 212d. Each optical detector 212a to 212d comprises here a pair of diodes, with a light emitting diode and an associated photodiode. The light emitting diode illuminates the discoloured liquid, and the reflected light is detected by the photodiode. Based on the colour of the reflected light, conclusions can then be drawn about the concentration of the substance in the body fluid.

A volumetric sensor arrangement 118 is also incorporated in the body of the measuring patch 100 for determining a volume of body fluid. In the present case, the volumetric sensor arrangement is a microfluidic channel 118, which is ring-shaped in this case. The body fluid excreted by the body is absorbed by the annular channel 118 and progressively fills it up over time. Conclusions can be drawn about the volume or flow rate of the excreted body fluid via the size of the partial volume of the channel 118, which is filled by the body fluid at a certain point in time.

Four electrodes 114a to 114d are distributed around the circumference of the measuring patch 100 at 90° intervals. These form a sensor array for bioelectrical impedance analysis, together with four respective contact electrode pairs 210a to 210d. The contact electrode pairs 210a to 210d are also distributed in a complementary manner at 90° intervals around the circumference of the back 204 of the readout unit 200. When the readout unit 200 is worn over the measuring patch 100, the contact electrode pairs 210a to 210d contact the respective extremities of the measuring patch electrodes 114a to 114d.

An optoelectronic sensor arrangement 216a, 216b is provided in the centre of the back 204 of the readout unit 200 for determining the pulse rate and/or the blood pressure. In the present case, this consists of two outer light emitting diodes 216a and a central photodiode 216b. The light emitting diodes 216a emit light which is at least partially reflected by the skin E of the user. The reflected light is detected by the photodiode 216b. Based on the change in the strength of the reflected light over time, the pulse rate and/or the blood pressure of the wearer of the measuring system S can be determined. In use, the optoelectronic sensor arrangement 216a, 216b is located above the access window 106 of the measuring patch 100. Accordingly, the optoelectronic sensor arrangement has direct access to the skin E.

Finally, two ECG electrodes 220a and 220b are arranged in the centre of the back 204. In use, these lie within the access window 106 of the measuring patch 100, so that they are in direct contact with the skin E.

In the second variant of FIGS. 4A-4B, the same sensor arrangements are provided in the centre of the back 204 as in the case of the first variant according to FIGS. 3A-3B. The microfluidic channel 118 is also found here in the measuring patch 100 for volume measurement. Two chemical sensor arrangements based on colorimetry are also provided (116a, 212a; 116b, 212b). The patch also has an electrode 114a associated with a complementary pair of contact electrodes 210a in the back 204. This can again be used, for example, to perform impedance measurements.

The special feature here is a mechanical pressure sensor 120 for measuring deformations of the skin E, which is integrated into the measuring patch 100. The pressure sensor 120 can be realised, for example, in the form of a serpentine conductor track whose electrical resistance changes with deformation. The change in resistance can be detected using a pair of electrodes 222 in the back 204 of the readout unit 200.

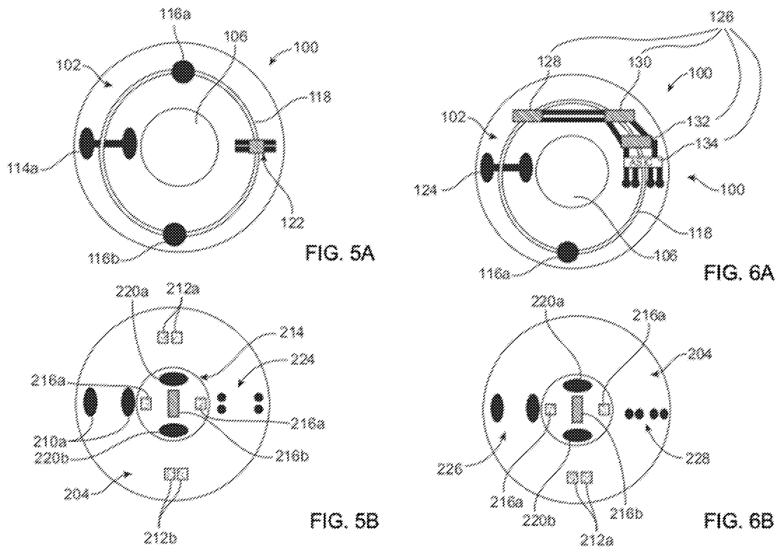

Also in the variant according to FIGS. 5A-5B, some of the same sensors are found as in the first variant of FIGS. 3A-3B. The special feature here is a sensor circuit 122 integrated into the body of the measuring patch 100. This is, for example, a microchip comprising an electrooptical sensor. The chip 122 is supplied with power by means of electrodes 224 in the back 204 of the readout unit 200. The electrodes 224 also serve to exchange data with the microchip 122.

In the fourth variant according to FIGS. 6A-6B, a special feature is an electrical sensor arrangement 124, 226 for determining the concentration of a substance in the body fluid. This electrical sensor arrangement comprises an electrode 124 arranged in the measuring patch, which is coated with a reagent. The measurement signal of the electrode is read out via a contact electrode pair 226 in the back 204 of the readout unit 200.

Another special feature is an electronic sensor circuit 126 integrated in the measuring patch 100. The sensor circuit 126 comprises here three sensor chips 128, 130 and 132 connected via a data and current bus. The three sensor chips are controlled and read out via an integrated microprocessor 134 (ASIC, Application Specific Integrated Circuit). In turn, electrodes 228 are located in the back 204 for power supply and data exchange with the electronic sensor circuit 126 located in the measuring patch 100.

In summary, the measurement system allows a good compromise to be found between the various requirements on so-called fitness or health trackers.

By combining a disposable patch with a durable body-worn readout unit, such as a wristwatch, the measurement system can be easily adapted to different fields of application.

This versatility results from the fact that different types of patches can be combined with the readout unit. The system can therefore be adapted to different fields of application in the fitness, wellness or health sector by using an appropriately adapted patch.

Another advantage is that the measurement system also allows real-time monitoring of physiological parameters. Since the adjustment is done by changing the patch, the operating costs are also manageable.

The measuring system, for example in the form of a measuring patch combined with a wristwatch, is also characterised by the fact that it is comfortable and discreet, and has an appearance that appeals to the consumer.

### LIST OF REFERENCE SIGNS

100 MEASURING PATCH
102 SENSOR SYSTEM
104 OUTER CONTOUR
106 ACCESS WINDOW
108 FRONT SIDE
110 BACK SIDE
111A, 111B CONTACT SURFACE
112 OUTPUT
114A TO 114D ELECTRODES
116A TO 116D FLUID CHAMBERS
118 MICROFLUIDIC CHANNEL
120 PRESSURE SENSOR
122 MICROCHIP
124 ELECTRODE
126 INTEGRATED CIRCUIT
128, 130,132 SENSOR CHIPS
134 MICROPROCESSOR
200 READOUT UNIT
202 FRONT
204 BACK
206A,206B CONTACT SURFACE
208 INPUT
210A TO 210D CONTACT ELECTRODES
212A TO 212D OPTICAL DETECTORS
214 SENSOR DEVICE
216A, 216B SENSOR DIODES
220A, 220B CONTACT ELECTRODES
222, 224, 226, 228 CONTACT ELECTRODES
S MEASURING SYSTEM
I INTERFACE
H HAND
G WRIST

1. A portable system for measuring and monitoring physiological parameters of a human by analysis of a body fluid, wherein the system comprises:
a consumable part in the form of a measuring patch to be adhered to the skin, the measuring patch being equipped with a sensor system configured to provide measured values of physiological parameters by body fluid analysis;
a body-worn readout unit suitable for continuous use, which is configured to read out and monitor the measured values supplied by the sensor system of the measuring patch,
wherein the readout unit is further configured to be worn over the measuring patch adhered to the skin and to read out and monitor the measured values in this position, and
wherein an access window is formed in the body of the measuring patch and a sensor device is provided in the readout unit, wherein the sensor device is located in use above the access window so that it has direct access to the skin via the access window.

2. The system according to claim 1, wherein:
the measuring patch comprises an output configured to generate the measured values;
the readout unit comprises an input complementary to the output configured to receive the measured values; and
the measuring patch and the readout unit are arranged in such a way that the output and the input are located opposite one another in the immediate vicinity during use on humans.

3. The system according to claim 18, wherein the interface is an optical and/or electrical interface.

4. The system according to claim 2, wherein the output and the input each comprise a complementary contact surface.

5. (canceled)

6. The system according to claim 1, further comprising a chemical sensor arrangement configured to determine the concentration of a substance in the body fluid.

7. The system according to claim 6, wherein the chemical sensor arrangement is based on colorimetry, wherein a colorimetric reagent is located in the measuring patch and an optical detector is located in the readout unit.

8. The system according to claim 7, wherein the optical detector comprises a light source configured to illuminate the reagent and a photodiode configured to detect the light reflected back from the reagent.

9. The system according to claim 1, further comprising an optoelectronic sensor arrangement.

10. The system according to claim 1, further comprising a volumetric sensor arrangement configured to determine a volume of the body fluid.

11. The system according to claim 10, wherein the volumetric sensor arrangement comprises a microfluidic channel in the measuring patch.

12. The system according to claim 1, further comprising an electrical sensor arrangement configured to determine the concentration of a substance in the body fluid.

13. The system according to claim 12, wherein the electrical sensor arrangement comprises an electrode arranged in the measuring patch which is coated with a reagent.

14. The system according to claim 1, further comprising a sensor arrangement for bioelectrical impedance analysis.

15. The system according to claim 14, wherein the sensor arrangement for bioelectrical impedance analysis comprises electrodes integrated in the readout unit.

16. The system according to claim 1, further comprising a mechanical pressure sensor configured to measure deformations of the skin.

17. The system according to claim 1, wherein the body fluid is sweat, blood, spit, or tear fluid.

18. The system according to claim 1, wherein the measuring patch and the readout unit are arranged in such a way that the output and the input make contact with one another in order to form an interface configured to transmit the measured values.

19. The system according to claim 9, wherein the optoelectronic sensor arrangement is configured to determine the pulse rate and/or the blood pressure.

20. The system according to claim 16, wherein the mechanical pressure sensor is integrated into the measuring patch.
